(19) United States
(12) Patent Application Publication
(10) Pub. No.: US 2022/0313394 A1
(43) Pub. Date: Oct. 6, 2022

(54) ADJUSTMENT MEANS FOR A MANDIBULAR ADVANCEMENT DEVICE

(71) Applicant: Bioanalytics Holdings Limited, Victoria (AU)

(72) Inventors: Owen MORGAN, New South Wales (AU); Guoping Yan, Victoria (AU); Anthony SASSE, Victoria (AU)

(73) Assignee: Bioanalytics Holdings Limited, Victoria (AU)

(21) Appl. No.: 17/753,287

(22) PCT Filed: Aug. 26, 2020
(86) PCT No.: PCT/AU2020/000087

§ 371 Date: Feb. 25, 2022

(30) Foreign Application Priority Data
Aug. 26, 2019 (AU) 2019903104

(51) Int. Cl. A61C 7/08; A61F 5/56; A61C 7/36; A61B 5/00; A61C 7/06

(52) U.S. Cl. CPC A61C 7/08; A61F 5/566; A61C 7/36; A61B 5/4818; A61B 5/0022; A61C 7/06

(57) ABSTRACT

The present invention provides adjustment means for a mandibular advancement device. The device includes both intraoral and extraoral portions. The intraoral portion includes a body portion. The body portion includes lower and upper plates. The lower plate is adapted to accommodate a pair of opposed arms that extend rearwardly and outwardly from the plate into the oral cavity when the device is worn. Each arm includes a posterior intraoral maxillary abutment surface. The lower plate includes a threaded hole on each side, adjacent a respective arm so that the threaded hole extends from the respective arm towards the edge of the plate. Each hole is adapted to receive a screw that is able to be screwed into the threaded hole, and when the screw has been sufficiently screwed, the inner end of the screw contacts its respective arm whereupon further turning of the screw forces the arm to move towards its opposite arm so that the position of the moved arm's posterior intraoral maxillary abutment surface is moved to a new position.

### FIELD OF THE INVENTION

This invention relates to mandibular advancement devices, particularly to devices worn by a person to prevent obstructive sleep apnoea, and in particular to adjustment means for the intraoral portion of that device.

### BACKGROUND OF THE INVENTION

A mandibular advancement device of the type referred to in this specification is already disclosed in WO 2019/071291 A1 and WO 2006/072147. The mandibular advancement device disclosed has both intraoral and extraoral portions. The present invention is mainly concerned with the intraoral portion of the device.

Due to wide variations in the size and shape of the oral cavity of users of these kinds of devices, and to keep manufacturing costs low, these devices are typically manufactured in three broad sizes, such as small, medium and large. However, there is a need for further adjustment within each of these broad sizes to maximise comfort and fit for each particular user, and also to maximise the effectiveness of the device.

One of the main problems surrounding the fit and comfort of the device is getting the engagement of the posterior maxillary abutment surfaces of each of the respective arms of the intraoral portion of the device in the correct position that conforms with the wearer's posterior maxillary characteristics. The posterior maxillary abutment surfaces are meant to loosely engage with the molars or premolars of the wearer and to resist any lateral movement of the device inside the oral cavity of the wearer. It is important that the device does not impose sufficient lateral force to the wearer's teeth so that tooth displacement may occur over time. Also, the imposition of unnecessary force will significantly negatively impact the wearer's comfort when wearing the device and may reduce the device's use and performance.

It is therefore an object of the present invention to provide adjustment means for the intraoral portion of a mandibular advancement device that at least mitigates some of the aforementioned problems.

### SUMMARY OF THE INVENTION

According to a first aspect, the present invention provides adjustment means for a mandibular advancement device, said device including both intraoral and extraoral portions, wherein the intraoral portion includes a body portion. The body portion includes lower and upper plates. The lower plate is adapted to accommodate a pair of opposed arms that extend rearwardly and outwardly from the plate into the oral cavity when the device is worn. Each arm includes a posterior intraoral maxillary abutment surface. The lower plate includes adjustment means on each side that are adapted to act upon a respective arm and move its position, relative to the opposing arm, so that the position of the moved arm's posterior intraoral maxillary abutment surface is moved to a new position.

Preferably, the lower plate includes a threaded hole on each side, adjacent a respective arm, so that the threaded hole extends from the respective arm towards the edge of the plate. Each hole is adapted to receive a screw that is able to be screwed into the threaded hole, and when the screw has been sufficiently screwed, the inner end of the screw contacts its respective arm whereupon further turning of the screw forces the arm to move relative to its opposite arm so that the position of the moved arm's posterior intraoral maxillary abutment surface is moved to a new position.

Preferably, each threaded hole in the bottom plate is recessed back from the outer edge and the opening of the threaded hole is respectively located within a vertical slot.

Preferably, the upper plate is adapted to overlay the bottom plate and join together with the bottom plate to form the body portion and encapsulate a portion of each of the arms within the body portion. The upper plate includes side tabs that extend substantially vertically down and are aligned with and adapted to be inserted into, and conform with the shape of, the vertical slot, thereby overlaying the entrance to the threaded hole and screw and forming a body portion with a substantially smooth outer surface.

Preferably, each side tab includes an aperture that is positioned so that it aligns with, and is concentric with, the threaded hole and the screw.

Preferably, each aperture is sized so that an adjustment tool, such as a screw driver or an Allen key is insertable, and is thereby able to engage with and act upon the screw, but is too small to enable the screw to be withdrawn through the aperture, thereby making the tab act as an abutment that prevents the screw from falling out of the body portion.

According to a second embodiment, the adjustment means are either electro-mechanical, or magnetic, thereby removing the need for an aperture on either the top or bottom plate, allowing the body to have a substantially continuous surface, allowing easier and more effective cleaning and sterilisation.

Preferably, each arm is integrally formed in either the top or bottom plate and fabricated out of a suitable elastomeric material.

Preferably, each arm is naturally biased with respect to the other with a maximum spread that the arms naturally rest at when not acted on by an external force, such as the impingement of the screw as it is operated on by the adjustment tool.

According to a third embodiment of the present invention, each arm is fabricated separately to either the top or bottom plate, and either the top or bottom plate includes a post portion that is profiled to resist rotation around the post, and the anterior end of each arm includes a hole that has a complimentary shape with, and is adapted to receive, the post portion within said hole, and this position is the natural rest position for each arm when the arm is not acted upon by an external force, such as the impingement of the screw as it is operated on by the adjustment tool.

According to a fourth embodiment of the present invention, each arm is fabricated separately to either the top plate or the bottom plate, and either the top plate or bottom plate includes a post portion with a circular profile, and the anterior end of each arm includes a hole that is adapted to receive the post portion and allow the arm to freely rotate about its respective post portion, and the body portion includes separate biasing means that act upon each respective arm and hold them at their maximum spread position when each arm is not acted upon by an external force, such as the impingement of the screw as it is operated on by the adjustment tool.

According to a fifth embodiment of the present invention the biasing means is a coil spring.

According to a sixth embodiment of the present invention, the biasing means is a leaf spring.

According to a seventh embodiment of the present invention, the biasing means is an elastomeric tab that is included in either the top plate or the bottom plate, and each said elastomeric tab is adapted to impinge against a respective arm.

Preferably, either the top or bottom plate includes an aperture, or window, that is associated with measurement increment indicators, and said measurement increments indicate the current position of each arm as an arm is forced to a new position under the influence of the grub screw.

Optionally, the body portion includes electronic sensor means that are adapted to sense the current position of each arm and feed that position data to wireless transmission means such as a Bluetooth transmitter.

Preferably, the data transmitted by the wireless transmission means are processed by an app on a portable computing device, such as a smart phone or tablet.

In another aspect, the present invention is a method of adjusting a mandibular advancement device in order to improve the fit and comfort of the wearer of said device, the method including the steps of:

a. selecting an appropriate device from a selection of small, medium and large size devices; and

b. inserting the device into the wearer's mouth; and

c. determining the fit and comfort of the device by bracing each intraoral maxillary abutment surface against an extramaxillary anterior maxillary abutment surface; and

d. receiving feedback from the wearer on the fit and comfort; and

e. using an appropriate tool to insert into an adjustment hole, if necessary, to turn the grub screw and force each respective arm to change position and thereby causing each posterior intraoral maxillary abutment surface to change position to provide a custom fit that best suits the wearer's mandibular.

Preferably, the method further includes the step of using the aperture, or window, and its associated measurement increment indicators to take note of the most comfortable setting for the particular wearer.

Optionally, the method further including the step of using the sensor means and wireless transmission means to transmit the position of each arm in an app on a portable computing device, such as a smart phone, and using the app to associate the optimal position of each arm to the wearer.

### BRIEF DESCRIPTION OF THE DRAWINGS

Examples of the invention will now be described with reference to the accompanying drawings in which:

FIG. 1 is an isometric view of the top side of the bottom plate.

FIG. 2 is another isometric view of the top of the bottom plate including a pair of grub screws.

FIG. 3 is an exploded view of the top and bottom plates.

FIG. 4 is an isometric view of the top and bottom plates joined together to form the body portion.

FIG. 5 shows the assembled substantially intraoral portion of the mandibular advancement device.

FIG. 6 shows a further embodiment of the invention so that the position of each arm can be measured.

FIG. 7 shows another embodiment of the invention where electronic sensors are included that sense the respective position of each arm and optionally transmit that data to an app on a portable computing device, such as a smart phone.

FIGS. 8 (a) and (b) show another preferred embodiment of the current invention where each arm is fabricated separately to the body portion and each arm is able to be placed upon a respective stake prefabricated into the bottom plate and is free to rotate about its respective stake, and the use of a natural leaf spring to hold each arm apart.

FIGS. 9 (a) and (b) show yet another preferred embodiment of the current invention where each arm is fabricated separately and is each able to be placed upon a respective stake that has been prefabricated into the bottom plate, however each arm is unable to rotate about its respective stake and thereby held in its maximum spread position.

FIGS. 10 (a) and (b) show another preferred embodiment of the invention using a pair of conventional coil springs to hold each respective arm in its maximum spread position.

### DETAILED DESCRIPTION OF THE PREFERRED EMBODIMENTS

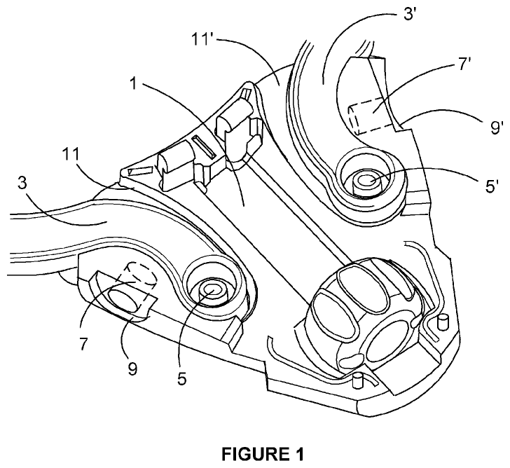

Turning firstly to FIG. 1, where we are shown an isometric view of the top of the bottom plate 1. The bottom plate 1 is a part of the body portion of the intraoral portion of a mandibular advancement device. The bottom plate 1 includes a pair of arms 3 and 3′ respectively. Each of the arms 3 and 3′ recede into the wearer's oral cavity. In this preferred embodiment, each arm is integrally formed in the bottom plate at 5 and 5′ respectively. Each of the arms 3 and 3′ are fabricated from a suitably resilient material that will elastically yield under the influence of an external force. Each arm rests at its maximum spread position with respect to its opposite arm, when not acted upon by an external force.

Adjacent to each arm is a threaded hole 7 and 7′ respectively. Each hole is reset back from the edge of the bottom plate 1 and each is substantially centrally located in a respective slot 9 and 9′. Each threaded hole 7 and 7′ is open at its innermost end.

Each arm 3 and 3′ respectively also has a gap 11 and 11′ into which each arm is able to move when acted upon by an external force in order to bring the arms closer together.

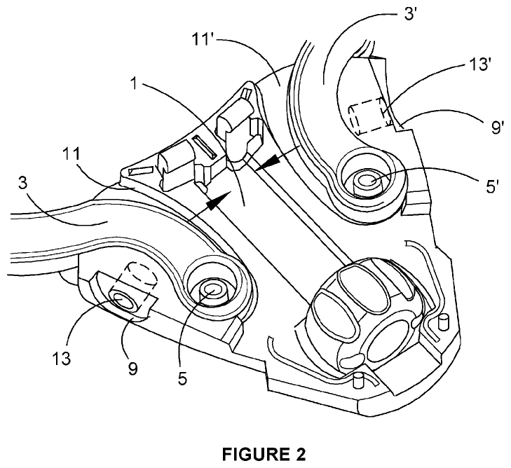

Turning to FIG. 2, we are shown that a pair of grub screws 13 and 13′ are adapted to be screwed into each respective threaded hole. When a tool such as a screw driver, or Allen key or the like is used, the grub screws may be turned. As each is turned, eventually the innermost end of the screw impinges upon the outermost side of its respective arm. This forces the arm to yield under the force applied by the screw. This causes the respective arm to move in the direction indicated by the arrows.

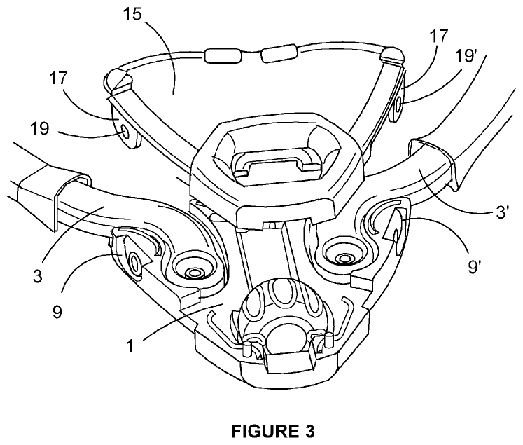

Turning to FIG. 3, we are shown the top plate 15 ready to be attached to the bottom plate 1. The top plate 15 includes a pair of downwardly extending tabs 17 and 17′ respectively. The size and shape of each of the tabs is selected so that when the top and bottom plates are connected, the tabs fit flush inside the respective slots 9 and 9′. Each tab 17 and 17′ includes a small aperture 19 and 19′. These apertures are adapted to be concentric with the hole and grub screws when the body portion is assembled. The aperture is sized so that a tool may pass through the aperture and engage the head of the grub screw, thereby enabling it to be turned in order to adjust the position of each arm. It is also sized so that the aperture is too small for the grub screw to be able to withdraw back through it if the screw is unwound within the threaded hole. Each respective tab 17 and 17′ thereby acts as an abutment that prevents it's respective grub screw from falling out of the body portion.

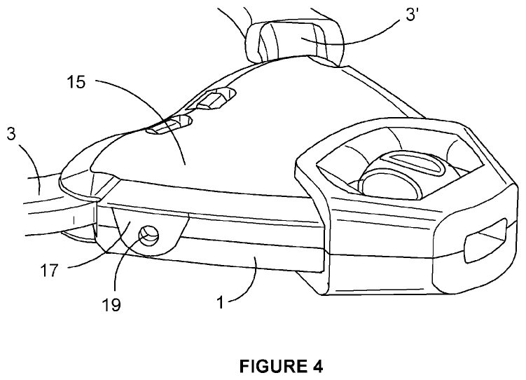

In FIG. 4, the body portion is assembled and illustrates how the tab 17 in the top plate 15 fits flush with the slot in the bottom plate 1.

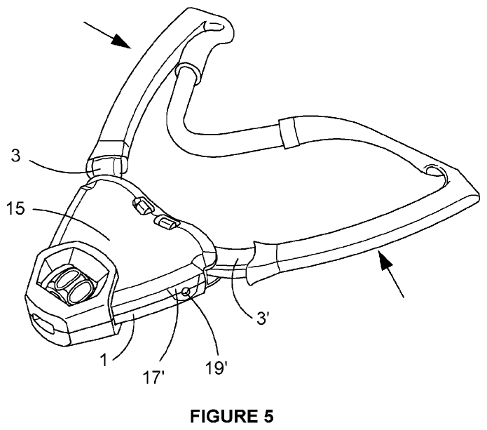

FIG. 5 shows the body portion comprising top plate 15 and bottom plate 1 assembled with other intraoral components of the mandibular advancement device. Adjustment of each respective grub screw by inserting a tool through aperture 19 and 19′ causes the arms 3 and 3′ to move closer together as shown by the arrows. In this embodiment, due to the natural resilience of the material from which the arms are fabricated, unscrewing the grub screw causes its respective arm to return to its normal rest position.

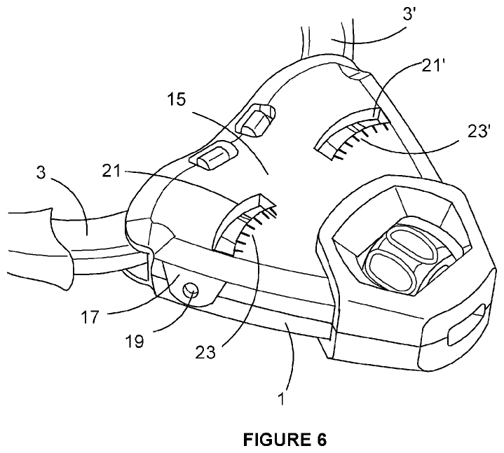

FIG. 6 shows an optional feature on the top plate 15. In this embodiment of the invention, a pair of windows 21 and 21′. Each window includes measurement graduations 23 and 23′. These offer the user a visual aid in determining the position of each arm 3 and 3′. The user can then determine the best arm position for that particular wearer of the device. This will particularly assist a user to setup subsequent devices for a particular wearer and manually set the arms into the initial best position for that particular person.

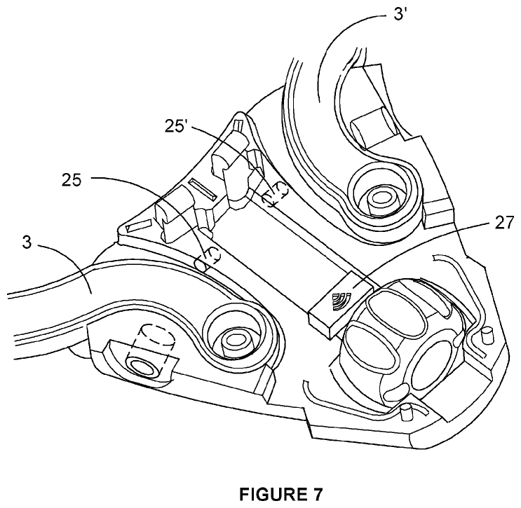

FIG. 7 shows another preferred embodiment of the invention. In this view, the body portion is fitted with electronic sensors 25 and 25 ‘. These sensors electronically sense the position of each arm 3 and 3’. The position data is then optionally transmitted via wireless means 27 to a portable computing device such as a smartphone. The smartphone has an app installed that links the position data to the user's account and logs the selected position of each arm that the wearer of the device finds to be the most comfortable. This data can then be used in the future when setting up ancillary or replacement devices for that particular wearer.

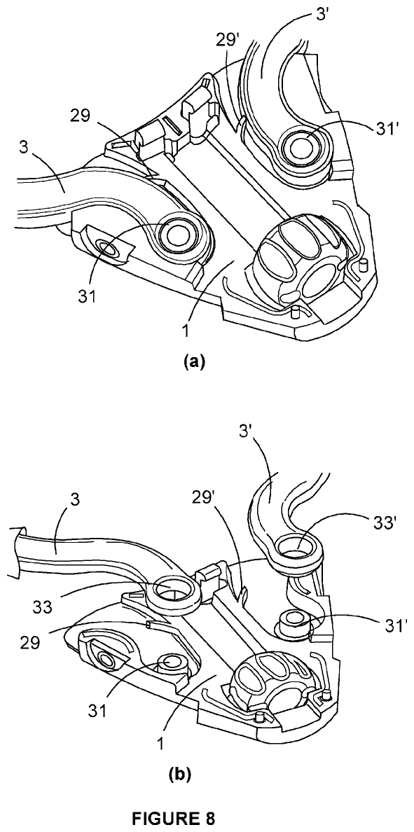

FIGS. 8 (a) and (b) show a preferred embodiment of the invention where a pair of natural leaf springs 29 and 29′ are prefabricated into the bottom plate 1. Each of the arms 3 and 3′ are fabricated separately to the bottom plate 1, and each arm includes a circular hole 33 and 33′ respectively at its anterior end which is designed to be placed upon a stake 31 and 31′ respectively. Each stake also has a matching circular profile. Each arm is then able to rotate about its respective stake, however each arm is biased into its maximum spread position by the force applied upon it by a respective leaf spring.

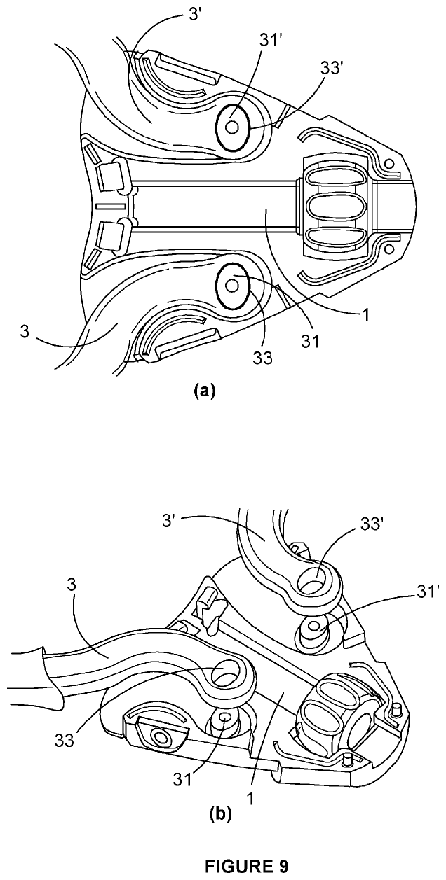

FIGS. 9 (a) and (b) show another preferred embodiment of the present invention. In this embodiment, each of the arms 3 and 3′ are fabricated separately to the bottom plate 1, however each of the holes 33 and 33′ are no longer circular in profile. Each of the stakes 31 and 31′ that a respective arm is placeable upon has a matching non-circular profile. The shape of the stake and the hole on the arm prevent the arm from rotating about the stake, and this holds each arm in its maximum spread position. Any force applied to a respective arm imposed by the grub screw will cause the arm to move towards its opposite arm. When the force is removed, the arm will move back to its maximum spread position under the influence of its own resilience.

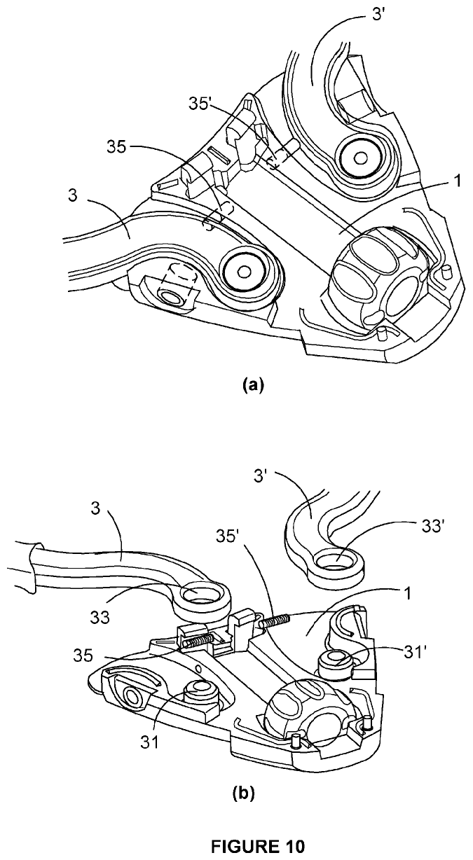

FIGS. 10 (a) and (b) show another preferred embodiment of the present invention. In this embodiment, each of the arms 3 and 3′ are fabricated separately to the bottom plate 1, and each is able to be placed upon a prefabricated stake 31 and 31′, and both the stakes and the holes in the arms have a circular profile, so that each arm is able to rotate around its respective stake, however a pair of springs 35 and 35′ are included in the assembly, and each spring applies a biasing force to a respective arm that holds the arm in its maximum spread position until acted upon by a respective grub screw.

So, from the aforementioned preferred embodiments, it can be seen that the present invention provides an easy way for a user to setup the intraoral portion of a mandibular advancement device so that it may have improved fit, comfort upon the wearer, and may also provide more efficient function.

While the above description includes the preferred embodiments of the invention, it is to be understood that many variations, alterations, modifications and/or additions may be introduced into the constructions and arrangements of parts previously described without departing from the essential features or the spirit or ambit of the invention. For example, the adjustment means in the preferred embodiment uses a threaded hole and associated grub screw, however other means of adjusting the position of the arm with respect to the other arm are possible, such as elector-mechanical means, or magnetic means. These should be considered within the scope of the present invention. Such means offer the ability to avoid the need for apertures in both the top and bottom plates, thereby making their respective surfaces continuous and thereby easier to clean and to sterilise between uses.

It will be also understood that where the word “comprise”, and variations such as “comprises” and “comprising”, are used in this specification, unless the context requires otherwise such use is intended to imply the inclusion of a stated feature or features but is not to be taken as excluding the presence of other feature or features.

The reference to any prior art in this specification is not, and should not be taken as, an acknowledgment or any form of suggestion that such prior art forms part of the common general knowledge.

1. Adjustment means for a mandibular advancement device, said device including both intraoral and extraoral portions, wherein the intraoral portion includes a body portion, and wherein the body portion includes lower and upper plates, and wherein the lower plate is adapted to accommodate a pair of opposed arms that extend rearwardly and outwardly from the plate into the oral cavity when the device is worn, and wherein each arm includes a posterior intraoral maxillary abutment surface, and wherein the lower plate includes adjustment means on each side that are adapted to act upon a respective arm and move its position, relative to the opposing arm so that the position of the moved arm's posterior intraoral maxillary abutment surface is moved to a new position.

2. The adjustment means as defined in claim 1 wherein each side of the lower plate includes a threaded hole, adjacent a respective arm, so that the threaded hole extends from the respective arm towards the edge of the plate, and wherein each hole is adapted to receive a screw that is able to be screwed into the threaded hole, and when the screw has been sufficiently screwed into said threaded hole, the inner end of the screw contacts its respective arm whereupon further turning of the screw forces the arm to move relative to its opposite arm so that the position of the moved arm's posterior intraoral maxillary abutment surface is moved to a new position.

3. The adjustment means as defined in claim 2 wherein each threaded hole in the bottom plate is recessed back from the outer edge and the opening of the threaded hole is respectively located within a vertical slot.

4. The adjustment means as defined in claim 3 wherein the upper plate is adapted to overlay the bottom plate and join together with the bottom plate to form the body portion and encapsulate a portion of each of the arms within the body portion.

5. The adjustment means as defined in claim 4 wherein the upper plate includes side tabs that extend substantially vertically down and are aligned with and adapted to be inserted into, and conform with the shape of, the vertical slot, thereby overlaying the entrance to the threaded hole and screw and forming a body portion with a substantially smooth outer surface.

6. The adjustment means as defined in claim 5 wherein each side tab includes an aperture that is positioned so that it aligns with, and is concentric with, the threaded hole and the screw.

7. The adjustment means as defined in claim 6 wherein each aperture is sized so that an adjustment tool, such as a screw driver or an Allen key, is insertable, and is thereby able to engage with and act upon the screw, but is too small to enable the screw to be withdrawn through the aperture, thereby making the tab act as an abutment that prevents the screw from falling out of the body portion.

8. The adjustment means as defined in claim 1 wherein each arm is integrally formed in either the top or bottom plate and fabricated out of a suitable elastomeric material.

9. The adjustment means as defined in claim 8 wherein each arm is naturally biased with respect to the other with a maximum spread that the arms naturally rest at when not acted on by an external force, such as the impingement of the screw as it is operated on by the adjustment tool.

10. The adjustment means as defined in claim 1 wherein each arm is fabricated separately to either the top or bottom plate, and either the top or bottom plate includes a respective post portion for each arm that is profiled to resist rotation around the post, and the anterior end of each arm includes a hole that has a complimentary shape with, and is adapted to receive, the post portion within said hole, and this position is the natural rest position for each arm when the arm is not acted upon by an external force, such as the impingement of the screw as it is operated on by the adjustment tool.

11. The adjustment means as defined in claim 1 wherein each arm is fabricated separately to either the top plate or the bottom plate, and either the top plate or bottom plate includes a respective post portion with a circular profile, and the anterior end of each arm includes a hole that is adapted to receive the post portion and allow the arm to freely rotate about its respective post portion, and the body portion includes separate biasing means that act upon each respective arm and hold them at their maximum spread position when each arm is not acted upon by an external force, such as the impingement of the screw as it is operated on by the adjustment tool.

12. The adjustment means as defined in claim 11 wherein the biasing means is at least one coil spring.

13. The adjustment means as defined in claim 11 wherein the biasing means is a leaf spring.

14. The adjustment means as defined in claim 11 wherein the biasing means is an elastomeric tab that is included in either the top plate or the bottom plate, and each said elastomeric tab is adapted to impinge against a respective arm.

15. The adjustment means as defined in claim 1 wherein either the top or bottom plate includes an aperture, or window, that is associated with measurement increment indicators, and said measurement increments indicate the current position of each arm as an arm is forced to a new position under the influence of the screw.

16. The adjustment means as defined in claim 15 wherein the body portion includes electronic sensor means that are adapted to sense the current position of each arm and feed that position data to wireless transmission means such as a Bluetooth transmitter.

17. The adjustment means as defined in claim 16 wherein the data transmitted by the wireless transmission means are processed by an app on a portable computing device, such as a smart phone or tablet.

18. A method of adjusting a mandibular advancement device including both intraoral and extraoral portions, wherein the intraoral portion includes a body portion, and wherein the body portion includes lower and upper plates, and wherein the lower plate is adapted to accommodate a pair of opposed arms that extend rearwardly and outwardly from the plate into the oral cavity when the device is worn, and wherein each arm includes a posterior intraoral maxillary abutment surface, and wherein the lower plate includes a threaded hole on each side, adjacent a respective arm, so that the threaded hole extends from the respective arm towards the edge of the plate, and wherein each hole is adapted to receive a screw that is able to be screwed into the threaded hole, and when the screw has been sufficiently screwed into said threaded hole, the inner end of the screw contacts its respective arm whereupon further turning of the screw forces the arm to move towards its opposite arm so that the position of the moved arm's posterior intraoral maxillary abutment surface is moved to a new position, wherein said method improves the fit and comfort of the wearer of said device, the method including the steps of:
a. selecting an appropriate device from a selection of small, medium and large size devices; and
b. inserting the device into the wearer's mouth; and
c. determining the fit and comfort of the device by bracing each intraoral maxillary abutment surface against an extramaxillary anterior maxillary abutment surface; and
d. receiving feedback from the wearer on the fit and comfort; and
e. using an appropriate tool, if necessary, to insert into the hole and thereby engaging the screw and turning it to force each respective arm to change position and thereby causing each posterior intraoral maxillary abutment surface to change position to provide a custom fit that best suits the wearer's mandibular.

19. The method as defined in claim 18 wherein either the top or bottom plate of the device further includes an aperture, or window, that is associated with measurement increment indicators, and said measurement increments indicate the current position of each arm as an arm is forced to a new position under the influence of the screw, said method further including the step of using the aperture, or window, and its associated measurement increment indicators to take note of the most comfortable setting for the particular wearer.

20. The method as defined in claim 19 wherein the body portion of the device further includes electronic sensor means that are adapted to sense the current position of each arm and feed that position data to wireless transmission means such as a Bluetooth transmitter, and wherein the data transmitted by the wireless transmission means are processed by an app on a portable computing device, such as a smart phone or tablet, said method further including the step of using the sensor means and wireless transmission means to transmit the position of each arm in an app on a portable computing device to associate the optimal position of each arm to the wearer.
